(19) United States
(12) Patent Application Publication
(10) Pub. No.: US 2023/0383317 A1
(43) Pub. Date: Nov. 30, 2023

(54) MEDIUM COMPOSITION INCLUDING ETHANOL FOR PRODUCTION OF 2,3-BUTANEDIOL FROM SYNTHETIC GAS AND 2,3-BUTANEDIOL PRODUCTION METHOD USING SAME

(71) Applicant: KOREA INSTITUTE OF SCIENCE AND TECHNOLOGY, Seoul (KR)

(72) Inventors: Hyunju OH, Seoul (KR); Youngsoon UM, Seoul (KR); Sun Mi LEE, Seoul (KR); Gyeongtaek GONG, Seoul (KR); Ja Kyong KO, Seoul (KR)

(21) Appl. No.: 18/249,379

(22) PCT Filed: Nov. 24, 2020
(86) PCT No.: PCT/KR2020/016710

§ 371 Date: Apr. 17, 2023

(30) Foreign Application Priority Data
Oct. 23, 2020 (KR) 10-2020-0138518

(51) Int. Cl. C12P 7/16; C12N 1/20

(52) U.S. Cl. CPC C12P 7/16; C12N 1/20

(57) ABSTRACT

The present invention relates to a composition for producing 2,3-butanediol by using ethanol and synthetic gas and a 2,3-butanediol production method. A composition according to an aspect of the present invention includes ethanol as an active ingredient. In the case where a 2,3-butanediol production strain is inoculated into a medium containing the composition and cultured and a synthetic gas is added thereto, 2,3-butanediol, which is a biofuel and a chemical substance, can be economically produced with ethanol serving as a substrate. In the metabolism process, the carbon flux can be concentrated on the production route of the target material, whereby the composition has an excellent effect of increasing the production efficiency of 2,3-butanediol and enhancing the productivity of 2,3-butanediol by controlling only fermentation conditions such as amounts of the synthetic gas or ethanol and stirring speeds of the medium.

### CROSS REFERENCE TO RELATED APPLICATIONS

The present application is a National Phase of International Application No. PCT/KR2020/016710 filed on Nov. 24, 2020, which claims the priority based on Korean Patent Application No. 10-2020-0138518 filed on Oct. 23, 2020, and the entire contents disclosed in the description and drawings of the corresponding applications are referenced in the present application.

### DESCRIPTION OF GOVERNMENT-SUPPORTED RESEARCH PROJECT

This research was conducted with the financial support from the National Research Foundation of Korea and the Ministry of Science and ICT. The project title is “Development of high-performance microorganism and fermentation technology for producing 1-hexanol from synthesis gas” (Project ID: 1345305463). The present specification relates to a composition for preparing 2,3-butanediol using ethanol and synthesis gas, and a method for preparing 2,3-butanediol using the same.

### TECHNICAL FIELD

### Background Art

Most of the energy used globally is produced form petrochemical resources. In order to overcome the limitation of these petrochemical resources, researches are actively conducted to develop renewable energy using various biomass. The biomass used for production of renewable energy is classified into first generation (e.g., corn), second generation (lignocellulosic biomass) and third generation (algae). The biomass has limitations in terms of cost raw materials, use of food resources, supply of raw materials, price, cultivation area, etc., and additional cost is incurred for pretreatment processes necessary for degradation into monosaccharides that can be uptaken by microorganisms, use of enzymes, etc. Use of a mixture gas of CO, CO₂ and H₂ can overcome the limitations of the biomass and provide an economical carbon source. In this regard, synthesis gas that can be produced from gasification of biomass and organic waste resources and byproduct gas and waste gas generated industrially in ironworks, etc. are drawing attentions.

More specifically, the synthesis gas is a mixture gas consisting of carbon monoxide, carbon dioxide and hydrogen, which is obtained through gasification of various carbon-based materials such as waste, coal, naphtha, heavy oil, etc. The emission of the synthesis gas into the atmosphere is one of the causes of global warming. In addition, about 13 million ton of byproduct gas is generated annually during smelting processes in ironworks in Korea, and 35-40% is emitted into the atmosphere as carbon monoxide. Efforts are being made for developing the policies and technologies for reducing carbon dioxide emission not only in Korea but also globally. The conversion of the synthesis gas produced during use of petroleum resources into valuable products can achieve supply of inexpensive raw materials and reduction of carbon dioxide (CO₂ fixation) at the same time.

2,3-Butanediol is an important chemical intermediate in many industrial fields. Methyl ethyl ketone (industrial solvent) and 1,3-butadiene (building block for synthetic rubber) are produced through dehydration of 2,3-butanediol. In addition, 2,3-butanediol is used for preparation of printing inks, synthetic flavors, softeners and humectants, and is used as a carrier of drugs and medications or as an antifreeze due to low freezing point. Although 2,3-butanediol and its derivatives can be synthesized chemically, the production cost is unstable due to the limited petroleum resources, rise in oil prices, etc. Therefore, biomass-based 2,3-butanediol production is being researched actively. The present disclosure aims at effective production of 2,3-butanediol using synthesis gas, which can overcome the economic efficiency and disadvantages of biomass.

The production of 2,3-butanediol through a microbial fermentation process using fermentable sugar as a carbon source has been researched until recently. Representative 2,3-butanediol-producing microorganisms include Klebsiella pneumoniae, Klebsiella oxytoca, Serratia marcescens, Enterobacter aerogenes, Paenibacillus polymyxa, etc. However, these microorganisms produce 2,3-butanediol using fermentable sugar such as glucose, xylose, etc. The biomass used by these microorganisms for the production of 2,3-butanediol include food resources such as corn (first-generation), lignocellolusic biomass (second-generation) and algae (third-generation). But, the second-generation biomass has the disadvantages of limitation in supply of raw materials such as cultivation area, etc., pretreatment cost, ineffective utilization of carbon source, etc., and the third-generation biomass has many problems in terms of ineffective utilization of carbon source, use of food resources, pretreatment cost, etc. In order to overcome these problems and produce 2,3-butanediol economically, research has been conducted on the production of 2,3-butanediol using an acetogen that can use synthesis gas or industrial byproduct gas as a carbon source.

The acetogen refers to a microorganism which produces acetate through an anaerobic fermentation process by using a synthesis gas mixture of CO, CO₂ and H₂ as a carbon and energy source or substrate. The acetogen converts synthesis gas to various substances (ethanol, acetate, butyrate, butanol, etc.) by the Wood-Ljungdahl pathway. Typically, Clostridium ljungdahlii, Clostridium autoethanogenum, Eubacterium limosum, Clostridium carboxidivorans P7, Peptostreptococcus productus, Butyribacterium methylotrophicum, etc. are known well. The major products of the acetogen are acetate and ethanol having two carbon atoms, and ethanol production has reached the stage of commercialization by global companies like LanzaTech, INROS Bio, etc. The acetogens capable of biosynthesizing 2,3-butanediol include Clostridium ljungdahlii, Clostridium autoethanogenum, Clostridium ragsdalei and Clostridium coskatii. It is known that they produce 2,3-butanediol from synthesis gas with a low efficiency of about 0.36 g/L or lower. Among them, C. autoethanogenum DSMZ 10061 produces mainly ethanol and acetate by using synthesis gas. Although it is reported to be able to produce 2,3-butanediol, the production yield of 2,3-butanediol from synthesis gas by the bacterium is very low [Kaspar Valgepea et al., H₂ drives metabolic rearrangements in gas-fermenting Clostridium autoethanogenum, Biotechnology for Biofuels Volume 11, Article number: 55 (2018)]. The inventors of the present disclosure thought that economical production of 2,3-butanediol would be possible if 2,3-butanediol could be produced with high efficiency from synthesis gas by using C. autoethanogenum, and have conducted researches.

When a microorganism uses a fermentable sugar as a carbon source, it is converted to acetyl-CoA via pyruvate through glycolysis, and acetate, ethanol, etc. are synthesized from the acetyl-CoA. When synthesis gas is used, acetyl-CoA is produced by the Wood-Ljungdahl pathway, and the acetate, ethanol, etc. are synthesized. For 2,3-butanediol production, when a fermentable sugar is used, the produced pyruvate is used as a precursor and converted to acetolactate and then to acetoin. Finally, the acetoin is converted to 2,3-butanediol by 2,3-butanediol dehydrogenase. That is to say, when a fermentable sugar is used, 2,3-butanediol can be synthesized before the pyruvate is converted to acetyl-CoA. In contrast, when synthesis gas is used, the production of ethanol, acetate, etc. from acetyl-CoA and the conversion to pyruvate occur competitively. Therefore, the productivity of 2,3-butanediol may be improved if the metabolism is focused to pyruvate (see FIG. 1 for the production pathways).

Therefore, the inventors of the present disclosure have aimed at effective production of 2,3-butanediol using C. autoethanogenum DSMZ 10061, which is known as an acetogenic bacterium capable of producing 2,3-butanediol, and at the same time, aimed at improving the productivity of 2,3-butanediol by focusing the metabolism of acetyl-CoA to pyruvate by C. autoethanogenum DSMZ 10061 by adding ethanol, which is a major product. This method allows the production of valuable 2,3-butanediol by using an inexpensive carbon source and adding a low-cost renewable additive. In addition, byproducts can be reduced by recycling the acetate and ethanol remaining after the separation of 2,3-butanediol for production of 2,3-butanediol.

### REFERENCES OF RELATED ART

### Patent Documents

(Patent document 1) KR 10-2016-0123108 A.
(Patent document 2) U.S. Pat. No. 8,673,603 B2.
(Patent document 3) WO 2009-1513425 A1.
(Patent document 4) KR 10-2012-0096756 A.
(Patent document 5) KR 10-2019-0088648 A.
(Patent document 6) WO 2007-117157 A1.
(Patent document 7) WO 2017-066498 A1.

### DISCLOSURE

### Technical Problem

The existing methods for producing 2,3-butanediol use a sugar or an acid as a carbon source (KR 10-2016-0123108 A, KR 10-2012-0096756 A and U.S. Pat. No. 9,771,603 B2). However, the method of using a sugar as a carbon source is inefficient in terms of cost and time due to expensive raw materials, pretreatment processes, use of enzymes, etc. In addition, the method of using an acid has the problem that bacterial growth is inhibited due to the addition of the acid.

In an aspect, the present disclosure is directed to providing a method for preparing 2,3-butanediol using ethanol and synthesis gas without the problem of ineffectiveness or inhibited bacterial growth. In addition, the present disclosure is directed to providing a method for improving the productivity of 2,3-butanediol from synthesis gas by adding ethanol.

### Technical Solution

In an aspect, the present disclosure provides a medium composition for preparing 2,3-butanediol, which contains ethanol as an active ingredient and is for culturing a bacterium which produces 2,3-butanediol from synthesis gas.

In another aspect, the present disclosure provides a method for preparing 2,3-butanediol, which includes: a step of inoculating a 2,3-butanediol-producing bacterium to a medium containing the composition; and a step of adding synthesis gas to the medium.

### Advantageous Effects

A composition according to an aspect of the present disclosure is a medium composition containing ethanol as an active ingredient. When a 2,3-butanediol-producing bacterium is cultured by inoculating to a medium containing the composition and synthesis gas is added, 2,3-butanediol, which is a biofuel and chemical, can be prepared economically from ethanol as a substrate. And, by focusing carbon flux to the target substance production pathway, the productivity of 2,3-butanediol can be improved. In addition, the productivity of 2,3-butanediol can be improved by controlling fermentation conditions such as the addition amount of the synthesis gas or ethanol and the stirring speed of the medium.

### BRIEF DESCRIPTION OF DRAWINGS

FIG. 1 schematically shows the metabolic pathway by which C. autoethanogenum DSMZ 10061 produces 2,3-butanediol using synthesis gas and a sugar according to an aspect of the present disclosure.

FIGS. 2A, 2B, and 2C show a result of fermenting synthesis gas with C. autoethanogenum DSMZ 10061 according to an aspect of the present disclosure. FIG. 2A shows the consumption of synthesis gas by the bacterium, FIG. 2B shows the growth of the bacterium and the change in pH, and FIG. 2C shows the amount of 2,3-butanediol (2,3-BDO), ethanol (EtOH) and acetate produced by the bacterium from synthesis gas after performing the fermentation for 168 hours.

FIGS. 3A, 3B, 3C, 3D, and 3E show a result of fermenting synthesis gas with C. autoethanogenum DSMZ 10061 according to an aspect of the present disclosure by varying stirring speed and the addition amount of synthesis gas. FIGS. 3A, 3B, 3C, and 3D show the consumption of synthesis gas and growth of the bacterium depending on the stirring speed and the addition amount of synthesis gas, and FIG. 3E shows the amount of 2,3-butanediol (2,3-BDO), ethanol (EtOH) and acetate produced by the bacterium from synthesis gas after performing the fermentation for 192 hours depending on the stirring speed and the addition amount of synthesis gas.

FIGS. 4A, 4B, 4C, 4D, 4E, and 4F show a result of comparing fermentation by C. autoethanogenum DSMZ 10061 according to an aspect of the present disclosure depending on repeated addition of synthesis gas or addition of acetate or ethanol. FIGS. 4A, 4B, and 4C show the consumption of synthesis gas, and FIGS. 4D, 4E, and 4F show the production of products depending on fermentation time.

FIGS. 5A, 5B, 5C, 5D, 5E, 5F, and 5G show a result of comparing fermentation by C. autoethanogenum DSMZ 10061 according to an aspect of the present disclosure depending on the addition amount and addition interval of ethanol and synthesis gas. FIGS. 5A, 5B, 5C, and 5D show the consumption of synthesis gas, FIGS. 5E and 5F show the production of 2,3-butanediol and ethanol depending on fermentation time, and FIG. 5G compares the change in the amount of 2,3-butanediol and ethanol after performing the fermentation for 48 hours.

### BEST MODE

Hereinafter, the present disclosure is described in detail.

In an aspect, the present disclosure provides a medium composition for preparing 2,3-butanediol, wherein the composition contains ethanol as an active ingredient, the 2,3-butanediol is prepared from synthesis gas, and the composition is for culturing a 2,3-butanediol-producing bacterium.

The medium composition for preparing 2,3-butanediol according to an aspect of the present disclosure may contain ethanol as an active ingredient. Ethanol and acetate are produced as main products by Clostridium autoethanogenum 10061 using synthesis gas. The bacterium produces ethanol and acetate from acetyl-CoA synthesized through metabolism of the synthesis gas. If a 2,3-butanediol-producing bacterium is inoculated to a medium containing the composition according to an aspect of the present disclosure and synthesis gas is added, the conversion of acetyl-CoA to pyruvate may be induced rather than to ethanol production, and thus the productivity of 2,3-butanediol may be improved by focusing the flux of supplied carbon to 2,3-butanediol production. Although the methods for producing 2,3-butanediol using an acid or a sugar have been proposed (KR 10-2016-0123108 A, KR 10-2012-0096756 A and U.S. Pat. No. 9,771,603 B2), these methods have the problems that they are economically inefficient and the productivity of 2,3-butanediol is decreased as bacterial growth is inhibited due to the added acid, as described above. However, the composition according to an aspect of the present disclosure can ensure economic efficiency because 2,3-butanediol can be produced using ethanol without decrease in productivity. In addition, the composition according to an aspect of the present disclosure is characterized in that 2,3-butanediol is produced through microbial fermentation as the microorganism consumes ethanol, unlike the conversion of ethanol to 2,3-butanediol using a chemical catalyst or enzyme (KR 10-2019-0088648 A). In addition, whereas the existing technology produces 2,3-butanediol using a sugar or produces 2,3-butanediol by supplying only carbon monoxide together with a sugar, the composition according to an aspect of the present disclosure enables the production of 2,3-butanediol at higher yield by using synthesis gas only. Furthermore, since the composition according to an aspect of the present disclosure allows the production of 2,3-butanediol using synthesis gas, not single gas, it provides superior effect in terms of time and cost because a process for separating the single gas (e.g., carbon monoxide) is unnecessary.

In an aspect of the present disclosure, the content of ethanol may be 1-25 g/L based on the total volume of the medium containing the medium composition. Specifically, the content of ethanol may be 1 g/L or more, 2 g/L or more, 3 g/L or more, 4 g/L or more, 5 g/L or more, 6 g/L or more, 7 g/L or more, 8 g/L or more, 9 g/L or more, 10 g/L or more, 11 g/L or more, 12 g/L or more, 13 g/L or more, 14 g/L or more, 15 g/L or more, 16 g/L or more, 17 g/L or more, 18 g/L or more, 19 g/L or more, 20 g/L or more, 21 g/L or more, 22 g/L or more, 23 g/L or more or 24 g/L or more, and 25 g/L or less, 24 g/L or less, 23 g/L or less, 22 g/L or less, 21 g/L or less, 20 g/L or less, 19 g/L or less, 18 g/L or less, 17 g/L or less, 16 g/L or less, 15 g/L or less, 14 g/L or less, 13 g/L or less, 12 g/L or less, 11 g/L or less, 10 g/L or less, 9 g/L or less, 8 g/L or less, 7 g/L or less, 6 g/L or less, 5 g/L or less, 4 g/L or less, 3 g/L or less or 2 g/L or less, based on the total volume of the medium containing the medium composition. However, the content of ethanol is not limited thereto as long as 2,3-butanediol can be produced without inhibiting microbial growth.

In an aspect of the present disclosure, the 2,3-butanediol-producing bacterium may be an acetogenic bacterium. The acetogenic bacterium is a bacterium which can utilize synthesis gas as a carbon source and energy source. During fermentation of a sugar by the acetogenic bacterium, 2,3-butanediol is synthesized from pyruvate through glycolysis, and the pyruvate is also converted to acetyl-CoA, yielding ethanol and acetate. When synthesis gas is used, acetyl-CoA is produced by the Wood-Ljungdahl pathway, and ethanol and acetate are produced. The acetyl-CoA is converted to pyruvate and then 2,3-butanediol may be produced. The pyruvate is essential for the production of 2,3-butanediol as an intermediate. When synthesis gas is fermented by the microorganism, it is necessary to convert acetyl-CoA to pyruvate rather than to products such as ethanol, acetate, etc. The supply amount of synthesis gas can be increased to increase the acetyl-CoA pool for conversion to pyruvate. However, it is necessary to control the supply amount of synthesis gas because the supply of an excessive amount of carbon monoxide can inhibit bacterial growth.

In addition, in an aspect of the present disclosure, the 2,3-butanediol-producing bacterium may be one or more selected from a group consisting of Clostridium ljungdahlii, Clostridium autoethanogenum, Clostridium ragsdalei, Clostridium coskatii, Eubacterium limosum, Clostridium carboxidivorans P7, Peptostreptococcus productus and Butyribacterium methylotrophicum, specifically Clostridium autoethanogenum, more specifically Clostridium autoethanogenum DSMZ 10061. However, any bacterium may be used without being limited thereto as long as it can produce 2,3-butanediol using synthesis gas. It has been only reported that the Clostridium autoethanogenum DSMZ 10061 can produce 2,3-butanediol using synthesis gas. But, according to an example of the present disclosure, 2,3-butanediol could be prepared with high efficiency by adding ethanol during fermentation of synthesis gas using the bacterium.

The 2,3-butanediol-producing bacterium according to the present disclosure may be any strain regardless of genetic recombination. It may be either a wild-type strain or a transformed strain. Specifically, it may be a wild-type strain, which has been biotechnologically engineered to overexpress or downregulate a gene as compared to a normal control group, or a genetically recombined transformed strain. The composition according to an aspect of the present disclosure may be for preparing 2,3-butanediol from synthesis gas. That is to say, the 2,3-butanediol may be prepared from synthesis gas. Specifically, the synthesis gas may contain carbon monoxide, carbon dioxide and hydrogen.

In an aspect of the present disclosure, the synthesis gas may be added continuously. Specifically, the synthesis gas may be added continuously such that the carbon monoxide contained in the synthesis gas is not depleted completely. More specifically, it may be added continuously such that the carbon monoxide exists above 0 kPa. Further more specifically, it may be added continuously such that the carbon monoxide exists at 1 kPa or higher. Even more specifically, it may be added continuously such that the carbon monoxide exists at 1 kPa or higher, 1.2 kPa or higher, 1.4 kPa or higher, 1.6 kPa or higher, 1.8 kPa or higher, 2 kPa or higher, 2.2 kPa or higher, 2.4 kPa or higher, 2.6 kPa or higher, 2.8 kPa or higher, 3 kPa or higher, 3.2 kPa or higher, 3.4 kPa or higher, 3.6 kPa or higher, 3.8 kPa or higher, 4 kPa or higher, 4.2 kPa or higher, 4.4 kPa or higher, 4.6 kPa or higher, 4.8 kPa or higher or 5 kPa or higher. According to an example of the present disclosure, the production of 2,3-butanediol was increased as the interval of the addition of synthesis gas was shorter. The production of 2,3-butanediol was increased when synthesis gas was added further before the partial pressure of carbon monoxide was decreased to 5 kPa or below (Experimental Example 4-1).

In an aspect of the present disclosure, the addition amount of synthesis gas may be 0.2-5 bar, specifically 0.2 bar or more, 0.3 bar or more, 0.4 bar or more, 0.5 bar or more, 0.6 bar or more, 0.7 bar or more, 0.8 bar or more, 0.9 bar or more, 1 bar or more, 1.1 bar or more, 1.2 bar or more, 1.3 bar or more, 1.4 bar or more, 1.5 bar or more, 1.6 bar or more, 1.8 bar or more, 2 bar or more, 3 bar or more or 4 bar or more, and 5 bar or less, 4 bar or less, 3 bar or less, 2.9 bar or less, 2.8 bar or less, 2.7 bar or less, 2.6 bar or less, 2.5 bar or less, 2.4 bar or less, 2.3 bar or less, 2.2 bar or less, 2.1 bar or less, 2 bar or less, 1.9 bar or less, 1.8 bar or less, 1.7 bar or less, 1.6 bar or less, 1.5 bar or less, 1.3 bar or less, 1.2 bar or less, 1 bar or less or 0.5 bar or less. However, the addition amount of synthesis gas is not limited thereto as long as carbon source necessary for microbial growth can be supplied and 2,3-butanediol can be produced without inhibition of microbial growth and without safety problem during the fermentation process owing to high pressure.

In an aspect of the present disclosure, the 2,3-butanediol may be produced by stirring the medium to which the synthesis gas has been added. Stirring speed may be 50-1000 rpm. Specifically, the stirring speed may be 50 rpm or higher, 60 rpm or higher, 70 rpm or higher, 80 rpm or higher, 90 rpm or higher, 100 rpm or higher, 110 rpm or higher, 120 rpm or higher, 130 rpm or higher, 140 rpm or higher, 150 rpm or higher, 160 rpm or higher, 180 rpm or higher, 200 rpm or higher, 300 rpm or higher, 400 rpm or higher, 600 rpm or higher or 800 rpm or higher, and 1000 rpm or lower, 800 rpm or lower, 600 rpm or lower, 400 rpm or lower, 200 rpm or lower, 190 rpm or lower, 180 rpm or lower, 170 rpm or lower, 160 rpm or lower, 150 rpm or lower, 140 rpm or lower, 130 rpm or lower, 120 rpm or lower, 110 rpm or lower, 100 rpm or lower, 90 rpm or lower, 80 rpm or lower or 60 rpm or lower. However, the stirring speed is not limited thereto as long as 2,3-butanediol can be produced by adding synthesis gas without inhibiting microbial growth.

In another aspect, the present disclosure provides a method for preparing 2,3-butanediol, which includes: a step of inoculating a 2,3-butanediol-producing bacterium to a medium containing a medium composition for preparing 2,3-butanediol, wherein the composition contains ethanol as an active ingredient, the 2,3-butanediol is prepared from synthesis gas, and the composition is for culturing the 2,3-butanediol-producing bacterium; and a step of adding synthesis gas to the medium.

In an aspect of the present disclosure, the composition containing ethanol may contain 1-25 g/L of ethanol. Specifically, the content of ethanol may be 1 g/L or more, 2 g/L or more, 3 g/L or more, 4 g/L or more, 5 g/L or more, 6 g/L or more, 7 g/L or more, 8 g/L or more, 9 g/L or more, 10 g/L or more, 11 g/L or more, 12 g/L or more, 13 g/L or more, 14 g/L or more, 15 g/L or more, 16 g/L or more, 17 g/L or more, 18 g/L or more, 19 g/L or more, 20 g/L or more, 21 g/L or more, 22 g/L or more, 23 g/L or more or 24 g/L or more, and 25 g/L or less, 24 g/L or less, 23 g/L or less, 22 g/L or less, 21 g/L or less, 20 g/L or less, 19 g/L or less, 18 g/L or less, 17 g/L or less, 16 g/L or less, 15 g/L or less, 14 g/L or less, 13 g/L or less, 12 g/L or less, 11 g/L or less, g/L or less, 9 g/L or less, 8 g/L or less, 7 g/L or less, 6 g/L or less, 5 g/L or less, 4 g/L or less, 3 g/L or less or 2 g/L or less, based on the total volume of the medium. However, the content of ethanol is not limited thereto as long as 2,3-butanediol can be produced without inhibiting microbial growth.

In an aspect of the present disclosure, the 2,3-butanediol-producing bacterium may be an acetogenic bacterium. The acetogenic bacterium is a bacterium which can utilize synthesis gas as a carbon source and energy source. During fermentation of a sugar by the acetogenic bacterium, 2,3-butanediol is synthesized from pyruvate through glycolysis, and the pyruvate is also converted to acetyl-CoA, yielding ethanol and acetate. When synthesis gas is used, acetyl-CoA is produced by the Wood-Ljungdahl pathway, and ethanol and acetate are produced. The acetyl-CoA is converted to pyruvate and then 2,3-butanediol may be produced. The pyruvate is essential for the production of 2,3-butanediol as an intermediate. When synthesis gas is fermented by the microorganism, it is necessary to convert acetyl-CoA to pyruvate rather than to products such as ethanol, acetate, etc. The supply amount of synthesis gas can be increased to increase the acetyl-CoA pool for conversion to pyruvate. However, it is necessary to control the supply amount of synthesis gas because the supply of an excessive amount of carbon monoxide can inhibit bacterial growth.

In addition, in an aspect of the present disclosure, the 2,3-butanediol-producing bacterium may be one or more selected from a group consisting of Clostridium ljungdahlii, Clostridium autoethanogenum, Clostridium ragsdalei, Clostridium coskatii, Eubacterium limosum, Clostridium carboxidivorans P7, Peptostreptococcus productus and Butyribacterium methylotrophicum, specifically Clostridium autoethanogenum, more specifically Clostridium autoethanogenum DSMZ 10061. However, any bacterium may be used without being limited thereto as long as it can produce 2,3-butanediol using synthesis gas. It has been only reported that the Clostridium autoethanogenum DSMZ 10061 can produce 2,3-butanediol using synthesis gas. But, according to an example of the present disclosure, 2,3-butanediol could be prepared with high efficiency by adding ethanol during fermentation of synthesis gas using the bacterium.

The 2,3-butanediol-producing bacterium according to the present disclosure may be any strain regardless of genetic recombination. It may be either a wild-type strain or a transformed strain. Specifically, it may be a wild-type strain, which has been biotechnologically engineered to overexpress or downregulate a gene as compared to a normal control group, or a genetically recombined transformed strain.

In an aspect of the present disclosure, the synthesis gas may contain carbon monoxide, carbon dioxide and hydrogen. The existing method of producing 2,3-butanediol using a sugar as a carbon source is disadvantageous in terms of pretreatment, raw material cost, utilization of food resources, etc. And, the preparation of 2,3-butanediol from single gas is uneconomical because an additional process of separating the single gas is necessary. In contrast, the preparation method according to an aspect of the present disclosure can solve the problem that occurs when a sugar is used as a carbon source because 2,3-butanediol can be prepared from ethanol and synthesis gas. In addition, it enables the production of 2,3-butanediol at high yield effectively in terms of time and cost because the process of separating single gas is unnecessary.

In an aspect of the present disclosure, the synthesis gas may be added continuously. Specifically, the synthesis gas may be added continuously such that the carbon monoxide contained in the synthesis gas is not depleted completely. More specifically, it may be added continuously such that the carbon monoxide exists above 0 kPa. Further more specifically, it may be added continuously such that the carbon monoxide exists at 1 kPa or higher. Even more specifically, it may be added continuously such that the carbon monoxide exists at 1 kPa or higher, 1.2 kPa or higher, 1.4 kPa or higher, 1.6 kPa or higher, 1.8 kPa or higher, 2 kPa or higher, 2.2 kPa or higher, 2.4 kPa or higher, 2.6 kPa or higher, 2.8 kPa or higher, 3 kPa or higher, 3.2 kPa or higher, 3.4 kPa or higher, 3.6 kPa or higher, 3.8 kPa or higher, 4 kPa or higher, 4.2 kPa or higher, 4.4 kPa or higher, 4.6 kPa or higher, 4.8 kPa or higher or 5 kPa or higher. According to an example of the present disclosure, the production of 2,3-butanediol was increased as the interval of the addition of synthesis gas was shorter. The production of 2,3-butanediol was increased when synthesis gas was added further before the partial pressure of carbon monoxide was decreased to 5 kPa or below (Experimental Example 4-1).

In an aspect of the present disclosure, the addition amount of synthesis gas may be 0.2-5 bar, specifically 0.2 bar or more, 0.3 bar or more, 0.4 bar or more, 0.5 bar or more, 0.6 bar or more, 0.7 bar or more, 0.8 bar or more, 0.9 bar or more, 1 bar or more, 1.1 bar or more, 1.2 bar or more, 1.3 bar or more, 1.4 bar or more, 1.5 bar or more, 1.6 bar or more, 1.8 bar or more, 2 bar or more, 3 bar or more or 4 bar or more, and 5 bar or less, 4 bar or less, 3 bar or less, 2.9 bar or less, 2.8 bar or less, 2.7 bar or less, 2.6 bar or less, 2.5 bar or less, 2.4 bar or less, 2.3 bar or less, 2.2 bar or less, 2.1 bar or less, 2 bar or less, 1.9 bar or less, 1.8 bar or less, 1.7 bar or less, 1.6 bar or less, 1.5 bar or less, 1.3 bar or less, 1.2 bar or less, 1 bar or less or 0.5 bar or less. However, the addition amount of synthesis gas is not limited thereto as long as carbon source necessary for microbial growth can be supplied and 2,3-butanediol can be produced without inhibition of microbial growth and without safety problem during the fermentation process owing to high pressure.

In an aspect of the present disclosure, the 2,3-butanediol may be produced by stirring the medium to which the synthesis gas has been added. Stirring speed may be 50-1000 rpm. Specifically, the stirring speed may be 50 rpm or higher, 60 rpm or higher, 70 rpm or higher, 80 rpm or higher, 90 rpm or higher, 100 rpm or higher, 110 rpm or higher, 120 rpm or higher, 130 rpm or higher, 140 rpm or higher, 150 rpm or higher, 160 rpm or higher, 180 rpm or higher, 200 rpm or higher, 300 rpm or higher, 400 rpm or higher, 600 rpm or higher or 800 rpm or higher, and 1000 rpm or lower, 800 rpm or lower, 600 rpm or lower, 400 rpm or lower, 200 rpm or lower, 190 rpm or lower, 180 rpm or lower, 170 rpm or lower, 160 rpm or lower, 150 rpm or lower, 140 rpm or lower, 130 rpm or lower, 120 rpm or lower, 110 rpm or lower, 100 rpm or lower, 90 rpm or lower, 80 rpm or lower or 60 rpm or lower. However, the stirring speed is not limited thereto as long as 2,3-butanediol can be produced by adding synthesis gas without inhibiting microbial growth.

The preparation method according to an aspect of the present disclosure may further include a step of further adding ethanol to the medium.

In an aspect of the present disclosure, in the step of further adding ethanol, 0.2-5 g/L of ethanol based on the total volume of the medium may be added once or repeatedly. The ethanol may be added repeatedly while continuously adding synthesis gas such that carbon monoxide exists in the synthesis gas. Specifically, 0.2-5 g/L of ethanol may be added once or more times to the medium. More specifically, 0.2 g/L or more, 0.4 g/L or more, 0.6 g/L or more, 0.8 g/L or more, 1 g/L or more, 1.2 g/L or more, 1.4 g/L or more, 1.6 g/L or more, 1.8 g/L or more, 2 g/L or more, 2.2 g/L or more, 2.4 g/L or more, 2.6 g/L or more, 2.8 g/L or more, 3 g/L or more, 3.2 g/L or more, 3.4 g/L or more, 3.6 g/L or more, 3.8 g/L or more, 4 g/L or more, 4.2 g/L or more, 4.4 g/L or more, 4.6 g/L or more or 4.8 g/L or more, and 5 g/L or less, 4.8 g/L or less, 4.6 g/L or less, 4.4 g/L or less, 4.2 g/L or less, 4 g/L or less, 3.8 g/L or less, 3.6 g/L or less, 3.4 g/L or less, 3.2 g/L or less, 3 g/L or less, 2.8 g/L or less, 2.6 g/L or less, 2.4 g/L or less, 2.2 g/L or less, 2 g/L or less, 1.8 g/L or less, 1.6 g/L or less, 1.4 g/L or less, 1.2 g/L or less, 1 g/L or less, 0.8 g/L or less, 0.6 g/L or less or 0.4 g/L or less of ethanol may be added once or more times to the medium. In addition, when the ethanol is added once or more times, the ethanol may be added repeatedly while continuously adding synthesis gas such that carbon monoxide exists in the synthesis gas during the preparation of 2,3-butanediol or during the culturing of the bacterium. More specifically, the ethanol may be added repeatedly while continuously adding synthesis gas such that carbon monoxide exists above 0 kPa. Further more specifically, the ethanol may be added repeatedly while continuously adding synthesis gas such that carbon monoxide exists at 1 kPa or higher. Even more specifically, the ethanol may be added repeatedly while continuously adding synthesis gas such that carbon monoxide exists at 1 kPa or higher, 1.2 kPa or higher, 1.4 kPa or higher, 1.6 kPa or higher, 1.8 kPa or higher, 2 kPa or higher, 2.2 kPa or higher, 2.4 kPa or higher, 2.6 kPa or higher, 2.8 kPa or higher, 3 kPa or higher, 3.2 kPa or higher, 3.4 kPa or higher, 3.6 kPa or higher, 3.8 kPa or higher, 4 kPa or higher, 4.2 kPa or higher, 4.4 kPa or higher, 4.6 kPa or higher, 4.8 kPa or higher or 5 kPa or higher.

In the preparation method according to an aspect of the present disclosure, 2,3-butanediol may be prepared by controlling fermentation conditions. The control of the fermentation conditions may include addition of ethanol, addition of synthesis gas, the addition amount of ethanol or synthesis gas, the interval of addition, stirring speed, etc. Specifically, 2,3-butanediol may be prepared using a bacterium which has been genetically recombined or not, e.g., a wild-type strain or a transformed strain, by controlling the fermentation conditions. In addition, 2,3-butanediol may be prepared without addition of a catalyst.

In addition, although there is a method of removing hydrogen from synthesis gas and then producing 2,3-butanediol, this method has the problem that cost is increased due to the process of removing hydrogen (U.S. Pat. No. 8,673,603 B2). The preparation method according to an aspect of the present disclosure is economically advantageous because it does not require a synthesis gas pretreatment process such as the removal of hydrogen.

In another aspect, the present disclosure may relate to a use of a medium composition containing ethanol as an active ingredient for culturing of a 2,3-butanediol-producing bacterium and preparation of 2,3-butanediol from synthesis gas.

In another aspect, the present disclosure may relate to a composition containing ethanol as an active ingredient, which is for culturing of a 2,3-butanediol-producing bacterium for preparation of 2,3-butanediol from synthesis gas.

Hereinafter, the present disclosure is described more specifically through experimental examples. However, the following experimental examples are provided only to help the understanding of the present disclosure and the scope of the present disclosure is not limited by them.

### [Experimental Example 1] Fermentation Using Synthesis Gas

In order to investigate the production of 2,3-butanediol by Clostridium autoethanogenum (C. autoethanogenum) DSMZ 10061 using synthesis gas as a substrate, fermentation was conducted as follows after supplying synthesis gas.

First, trace elements were added to 1 L of a PETC medium (ATCC medium 1754) containing 2 g of yeast extract, 2 g of ammonium chloride (NH₄Cl), 0.08 g of calcium chloride (CaCl₂)·2H₂O), 0.4 g of magnesium sulfate (MgSO₄·7H₂O), 0.2 g of potassium chloride (KCl), 0.2 g of potassium phosphate (KH₂PO₄), 0.01 g of manganese sulfate (MnSO₄·H₂O), 0.002 g of sodium molybdate (NaMoO₄·2H₂O) and 0.2 g of cysteine. 50 mM 2-(N-morpholino)ethanesulfonic acid (MES) was added for pH buffering during fermentation, and the initial pH of the medium was adjusted to 6 using 2 M potassium hydroxide (KOH).

For batch culture, 20 mL of the medium was added to a 157-mL serum bottle and synthesis gas was added at 1.5 bar after inoculating C. autoethanogenum DSMZ 10061. The synthesis gas consisted of carbon monoxide (CO), carbon dioxide (CO₂) and hydrogen (H₂) at a ratio of 3:3:4. Culturing was conducted in a shaking incubator at 37° C. and 150 rpm, and gas consumption, bacterial growth, pH change and products were analyzed with time.

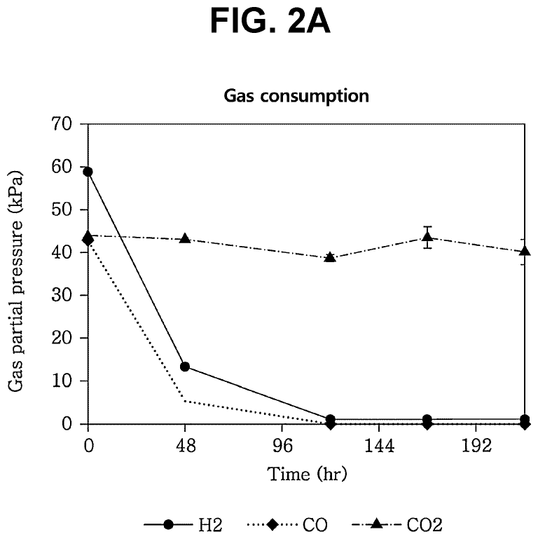

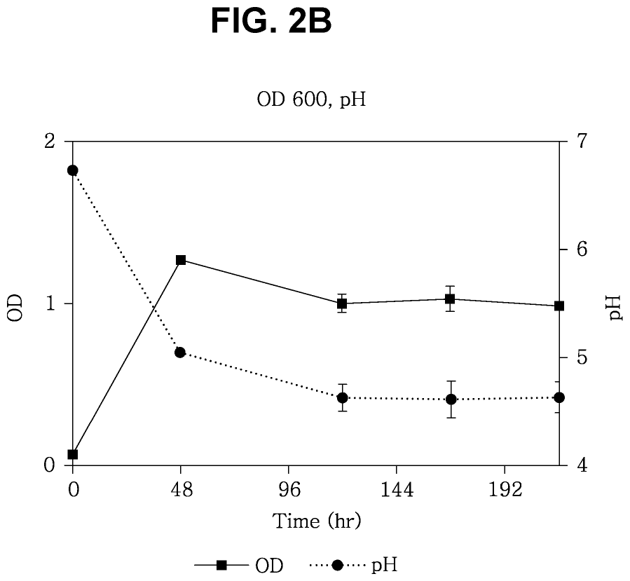

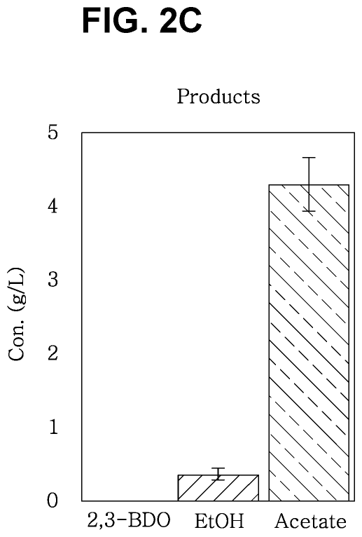

The change in the concentration of carbon monoxide (CO), carbon dioxide (CO₂) and hydrogen (H₂) in the synthesis gas depending on time was measured using a thermal conductivity detector (TCD; Agilent Technologies 6890N, USA), and the growth of the microorganism was analyzed by measuring absorbance at 600 nm with a spectrophotometer (Cary 60, Agilent Technologies, CA, USA). The products were analyzed with a gas chromatograph (Agilent model 6890N gas chromatograph). The result is shown in FIGS. 2A-2C.

As shown in FIG. 2A, when 1.5 bar of synthesis gas (CO:CO₂:H₂=3:3:4) was supplied, the supplied carbon monoxide and hydrogen were consumed completely within 120 hours of fermentation, whereas the amount of carbon dioxide did not change significantly. This is because carbon monoxide is converted to carbon dioxide and carbon dioxide and hydrogen are consumed together by the Wood-Ljungdahl pathway.

In addition, as shown in FIG. 2B, the bacterium grew fast during the fermentation, but the growth rate was lower than in fermentation using a sugar. This is because, whereas energy (ATP) necessary for the bacterial growth is supplied from glycolysis and acetate production when a sugar is used, the energy (ATP) is supplied from acetate production only when synthesis gas is used. pH was decreased with the bacterial growth. This is because acetate is produced to supply the energy necessary for the bacterial growth.

As shown in FIG. 2C, the products produced through the fermentation were identified as ethanol and acetate, and 2,3-butanediol was not observed. This is because of acetyl-CoA is converted to acetate or ethanol, rather than to pyruvate. The addition amount of synthesis gas should be controlled to overcome this problem.

### [Experimental Example 2] Production of 2,3-Butanediol Depending on Stirring Speed and Addition Amount of Synthesis Gas

During fermentation, the dissolution rate of synthesis gas in the medium changes depending on the stirring speed and addition amount of the synthesis gas and it affects the bacterial growth and produced products. As seen from FIG. 2C, only ethanol and acetate were produced from the fermentation and 2,3-butanediol was not produced when only synthesis gas was added. Therefore, the production of 2,3-butanediol by C. autoethanogenum DSMZ 10061 was investigated by varying fermentation conditions (stirring speed and addition amount of synthesis gas).

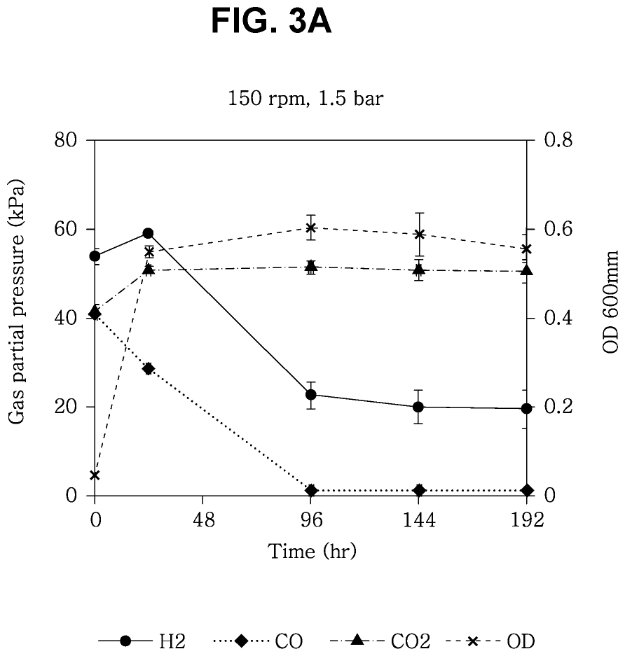

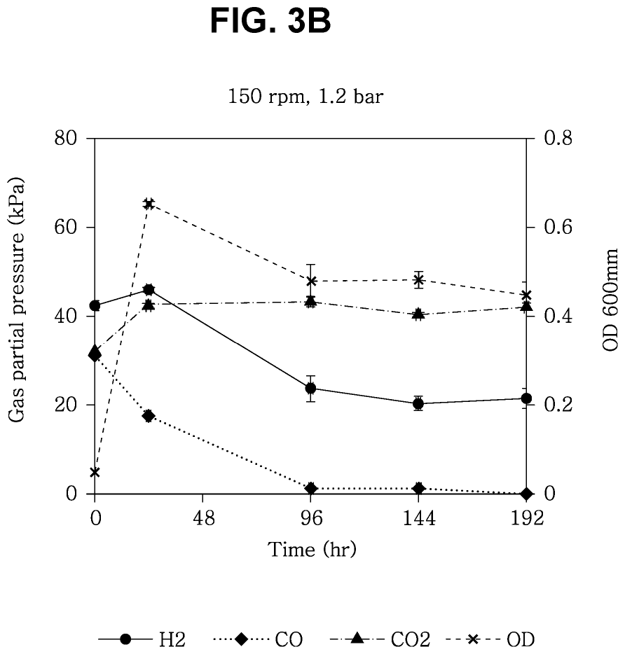

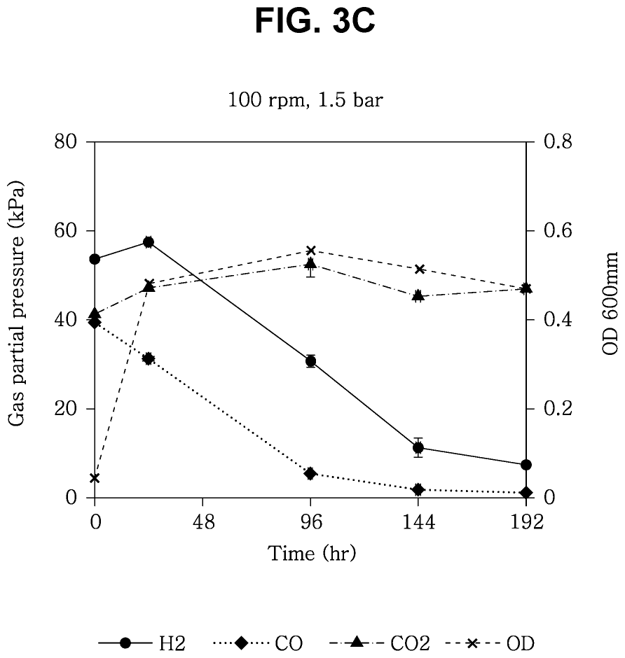

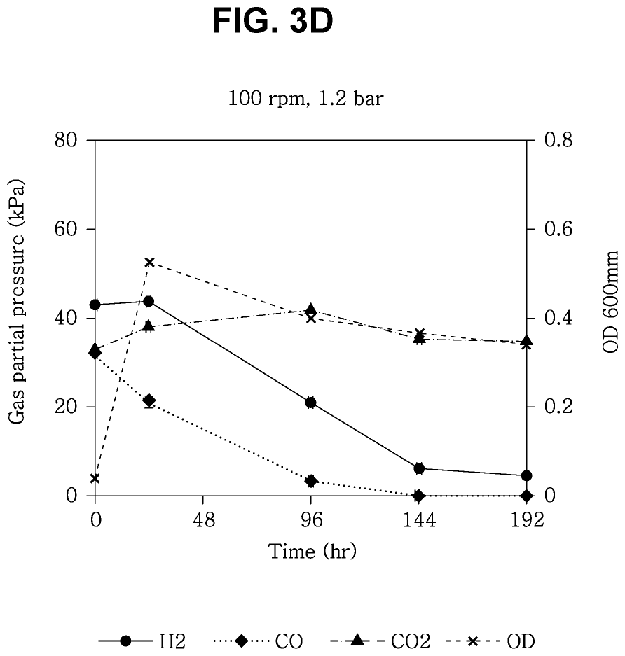

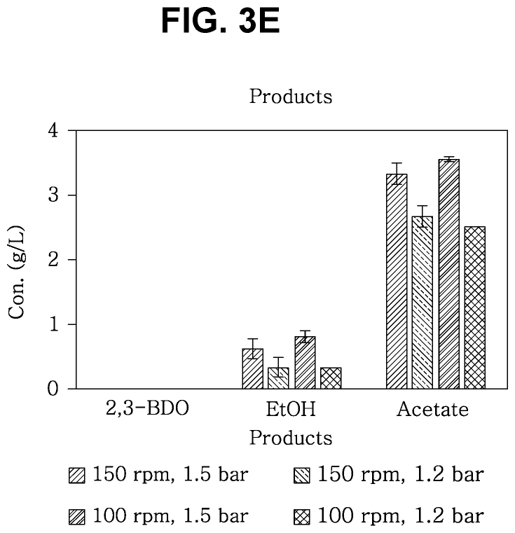

First, fermentation by C. autoethanogenum DSMZ 10061 was conducted in the same manner as in Experimental Example 1, by adding synthesis gas at 1.5 bar or 1.2 bar and varying the stirring speed as 150 rpm or 100 rpm. The result is shown in FIGS. 3A-3E.

As shown in FIGS. 3A-3D, when the addition amount of synthesis gas was 1.5 bar or 1.2 bar, the added carbon monoxide and hydrogen were consumed at the same time and the carbon monoxide was consumed completely. In addition, when the stirring speed was 150 rpm, the dissolution rate of carbon monoxide in the medium was increased and, thus, the carbon monoxide was consumed faster than when the stirring speed was 100 rpm. When the stirring speed was decreased from 150 rpm to 100 rpm, the dissolution rate of the synthesis gas in the medium was decreased and, therefore, the bacterial growth was less inhibited by the carbon monoxide. When the stirring speed was 100 rpm, the carbon monoxide was consumed slowly, but the consumption of hydrogen was increased and the added hydrogen was depleted almost when the fermentation was finished.

In addition, as shown in FIG. 3E, the major products were ethanol and acetate when the fermentation was finished, and the production of ethanol and acetate was increased as the addition amount of the synthesis gas was increased. When the addition amount of synthesis gas was 1.2 bar, the stirring speed had no significant effect on the production of ethanol and acetate. However, when the addition amount of synthesis gas was 1.5 bar, ethanol production was increased from 0.59 g/L to 0.76 g/L and acetate production was increased from 3.41 g/L to 3.68 g/L when the stirring speed was decreased from 150 rpm to 100 rpm. This is because the dissolution rate of carbon monoxide was changed due to the stirring speed and, thus, hydrogen consumption was increased. However, 2,3-butanediol could not be prepared by changing the stirring speed and addition amount of synthesis gas only.

### [Experimental Example 3] Production of 2,3-Butanediol Depending on Continuous Addition of Synthesis Gas and Addition of Acetate or Ethanol

Based on the results of Experimental Examples 1 and 2, it was investigated whether 2,3-butanediol is produced when synthesis gas is added continuously and acetate or ethanol is added in the early stage of fermentation.

### [Experimental Example 3-1] Production of 2,3-Butanediol Depending on Continuous Addition of Synthesis Gas

It was investigated whether the continuous addition of synthesis gas provides sufficient energy (ATP) for the bacterium and allows production of 2,3-butanediol through conversion of acetyl-CoA to pyruvate.

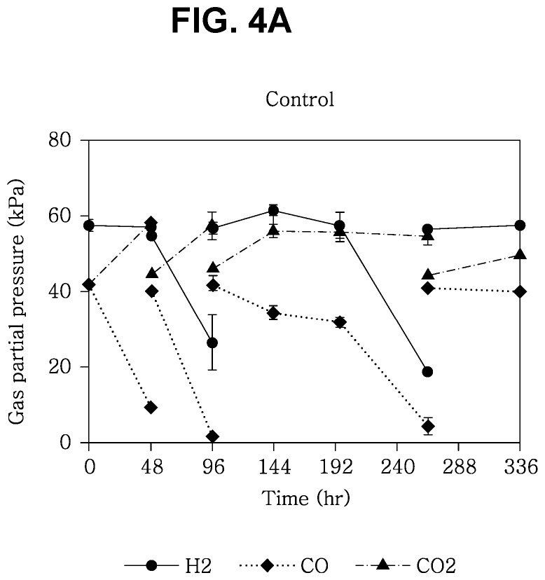

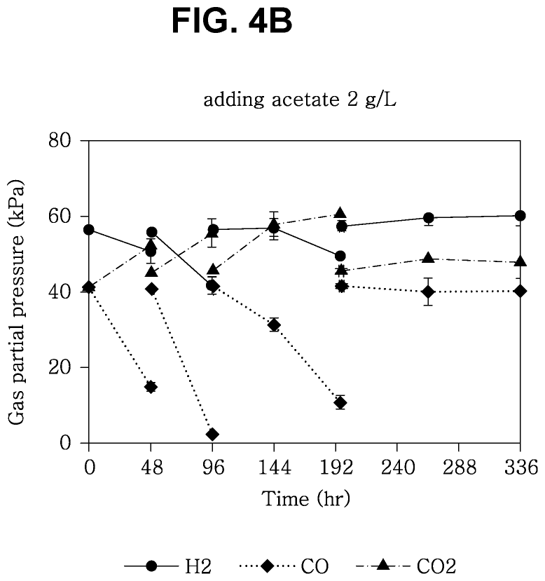

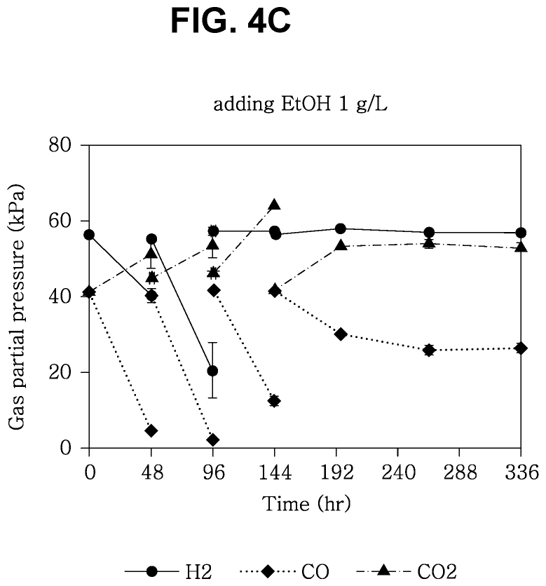

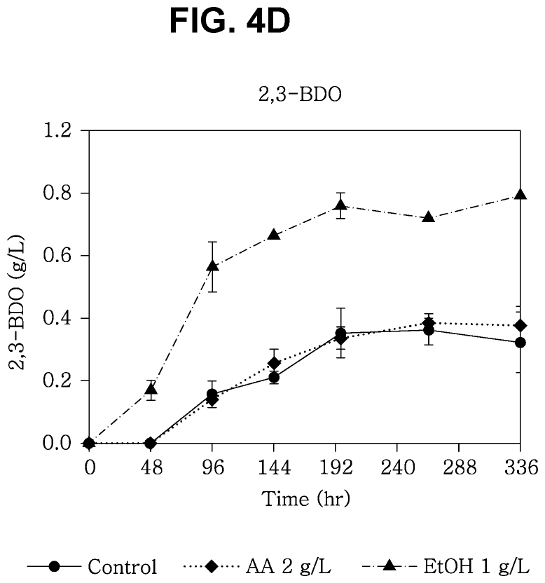

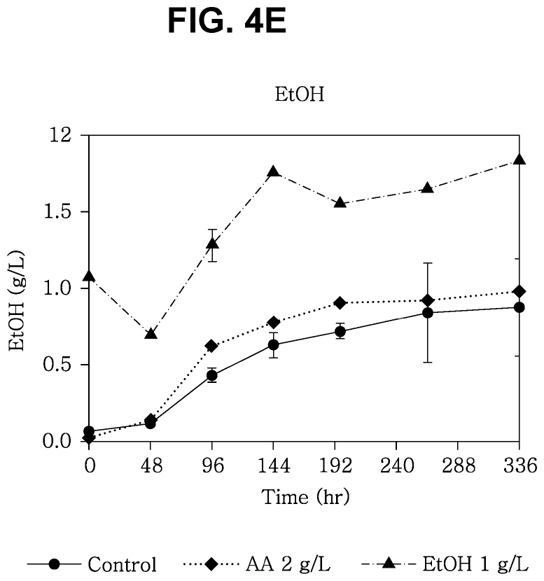

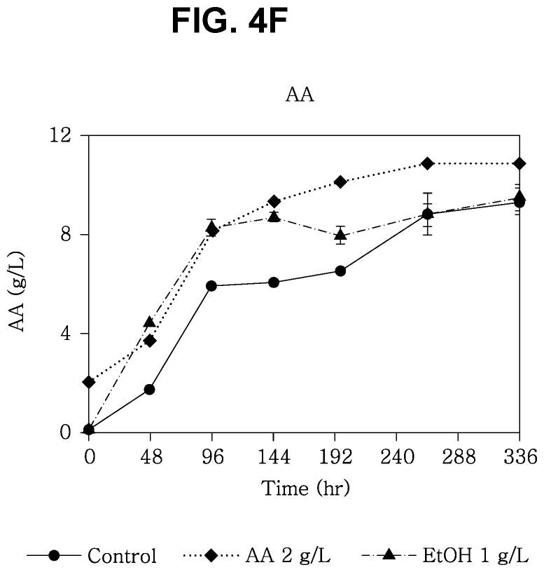

First, fermentation was conducted using C. autoethanogenum DSMZ 10061 in the same manner as in Experimental Example 1, except that the synthesis gas of the same composition was added again before carbon monoxide was consumed completely (hereinafter, Example 1-1) (Control in FIGS. 4A-4F). The result is shown in FIGS. 4A and 4D-4F.

As shown in FIG. 4A, when the synthesis gas was supplied continuously during the fermentation, carbon monoxide and hydrogen were consumed at the same time. In addition, as shown in FIG. 4D, 0.32 g/L of 2,3-butanediol was added when the fermentation was finished (fermentation for 336 hours). It is though that the continuous addition of synthesis gas provides sufficient energy for the bacterium and, thus, allows production of 2,3-butanediol through conversion of acetyl-CoA to pyruvate. Through this, it was confirmed that a sufficient amount of synthesis gas should be added for producing 2,3-butanediol.

### [Experimental Example 3-2] Production of 2,3-Butanediol Depending on Addition of Acetate or Ethanol

It was investigated whether the addition of acetate or ethanol, which are the major products of the Wood-Ljungdahl pathway, can increase the production of 2,3-butanediol by focusing the metabolism of acetyl-CoA to pyruvate rather than acetate or ethanol.

First, fermentation was conducted using C. autoethanogenum DSMZ 10061 in the same manner as in Experimental Example 3-1, except that 2 g/L of acetate was added to the medium in the early stage of fermentation using sodium acetate (C₂H₃NaO₂) (hereinafter, Example 1-2) (adding acetate 2 g/L in FIG. 4B, AA 2 g/L in FIGS. 4D-4F) and adding 1 g/L of ethanol to the medium in the early stage of fermentation (hereinafter, Example 1-3) (adding EtOH 1 g/L in FIG. 4C, EtOH 1 g/L in FIGS. 4D-4F). The result is shown in FIGS. 4B-4F.

As shown in FIGS. 4B and 4C, the consumption of carbon monoxide was not decreased by the addition of acetate or ethanol.

Although it was expected that the addition of acetate in the early stage of fermentation (Example 1-2) would lead to decreased production of acetate and, thus, increased production of ethanol or 2,3-butanediol as compared to when the acetate was not added (Example 1-1), no significant difference was observed in the consumption of synthesis gas and produced products, as shown in FIGS. 4B and 4D-4F. It has been reported that the addition of acetate to an aerobic microorganism that produces 2,3-butanediol using a sugar leads to increased production of C₂ or higher products because the added acetate is converted to 2,3-butanediol by CoA transferase (KR 10-2016-0123108 A). However, for the acetogenic bacterium, the addition of acetate in the early stage of fermentation increased neither the conversion of acetyl-CoA to pyruvate nor the production of 2,3-butanediol. This may be because the carbon flux is focused to the acetate production pathway because the microorganism can obtain the energy (ATP) necessary for growth from acetate production during the metabolism using synthesis gas. Regardless of the addition of acetate, the microorganism obtains the energy (ATP) necessary for growth by producing acetate. Therefore, the production amount of products was similar for Example 1-1 without addition of acetate (Control in FIGS. 4A-4F; 2,3-butanediol 0.38 g/L, ethanol 0.88 g/L, acetate 9.34 g/L) and Example 1-2 with addition of acetate (AA 2 g/L in FIGS. 4B and 4D-4F; 2,3-butanediol 0.38 g/L, ethanol 0.98 g/L, acetate 8.83 g/L (acetate at 336 hours after fermentation-acetate at 0 hour)).

As shown in FIGS. 4D and 4E, when ethanol was added, the added ethanol was decreased fast by 0.38 g/L (35.19% of added ethanol) within 48 hours of fermentation and 2,3-butanediol was produced fast from the early stage of fermentation. In addition, 0.80 g/L of 2,3-butanediol was produced for Example 1-3 with ethanol added (EtOH 1 g/L in FIG. 4D) at the end of the fermentation, which was improved by 2.5 times as compared to Example 1-1 with no ethanol added (Control in FIG. 4D). That is to say, the addition of ethanol increased reducing power (NADH) through conversion of the added ethanol to acetyl-CoA by the microorganism, without affecting the consumption of synthesis gas, and the increased reducing power (NADH) resulted in improved production of 2,3-butanediol. In contrast, for Example 1-1 with no ethanol added, it is thought that the reducing power was used competitively for the production of 2,3-butanediol and ethanol rather than being focused to 2,3-butanediol production. When only ethanol was added in the absence of synthesis gas, microbial growth was not observed and no 2,3-butanediol was produced. Accordingly, it was confirmed that the addition of ethanol during the fermentation of synthesis gas has an effect of shifting the carbon flux toward the 2,3-butanediol production pathway.

### [Experimental Example 4] Change in Production of 2,3-Butanediol Depending on Addition Amount of Ethanol and Continuous Addition of Synthesis Gas

From Experimental Example 3, it was confirmed that the addition of ethanol in the early stage of fermentation increases the consumption of ethanol and the production of 2,3-butanediol. Experiment was conducted as follows to investigate the effect of the addition amount of ethanol and the continuous supply of synthesis gas on the production amount of 2,3-butanediol.

### [Experimental Example 4-1] Change in Production of 2,3-Butanediol Depending on Continuous Addition of Synthesis Gas

In order to investigate the change in the production of 2,3-butanediol depending on the maintenance of the amount of carbon monoxide in synthesis gas during fermentation using the synthesis gas, fermentation was conducted using C. autoethanogenum DSMZ 10061 in the same manner as in Experimental Example 3-1, except that the synthesis gas was supplied additionally before the amount of carbon monoxide in the synthesis gas decreased to 5 kPa or below.

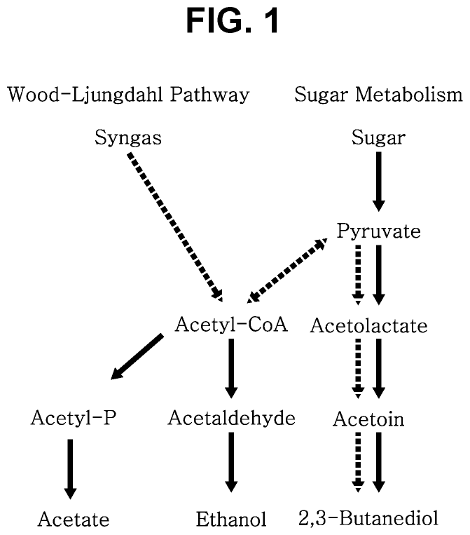

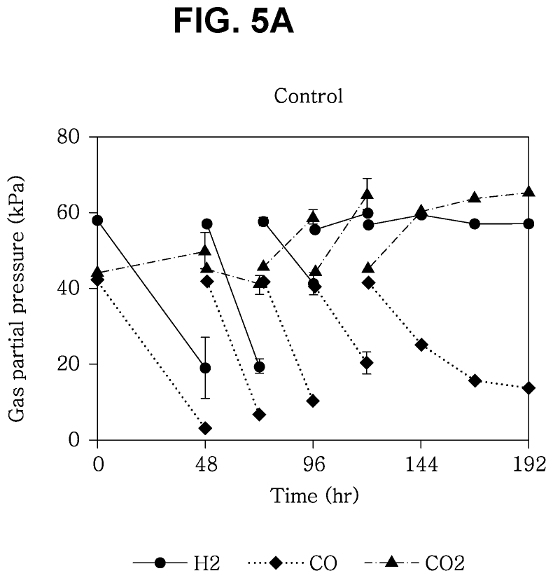

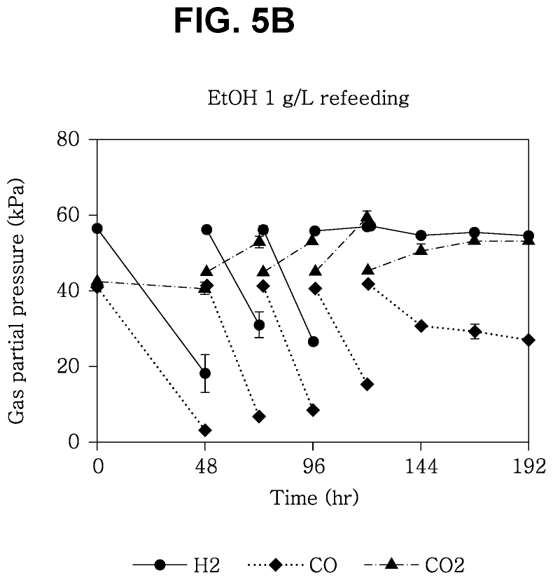

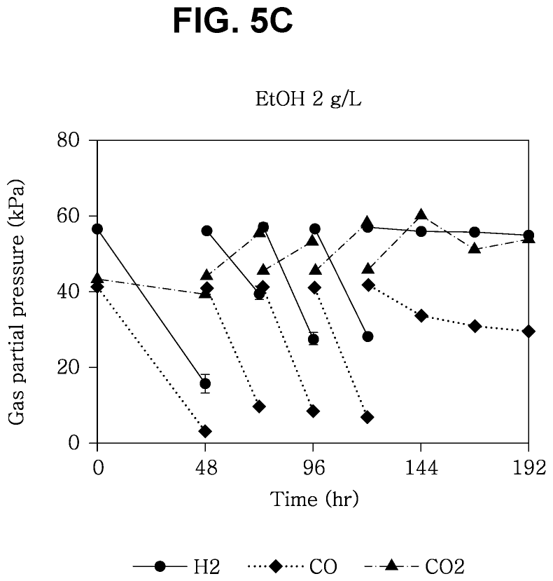

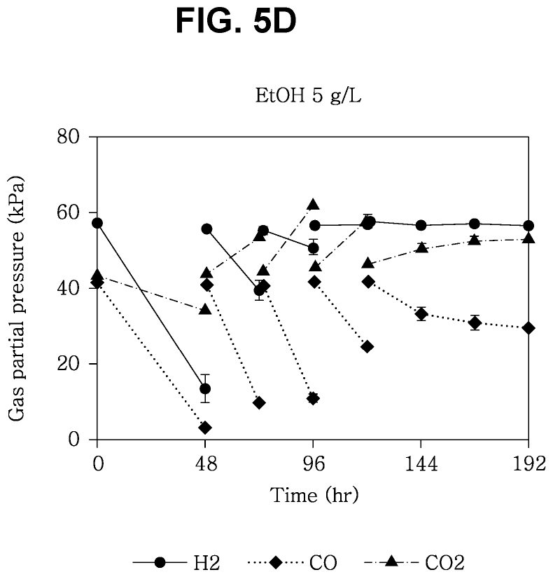

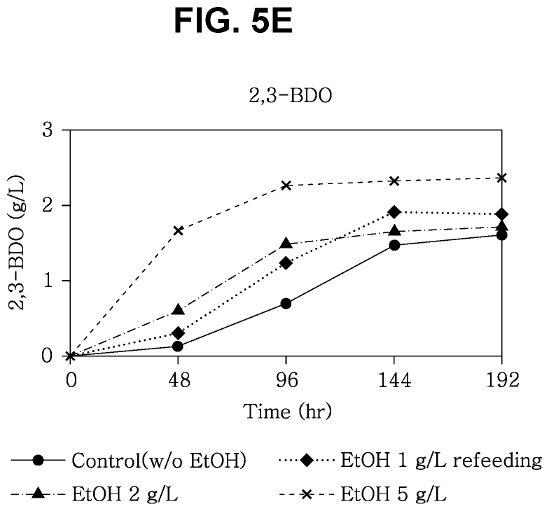

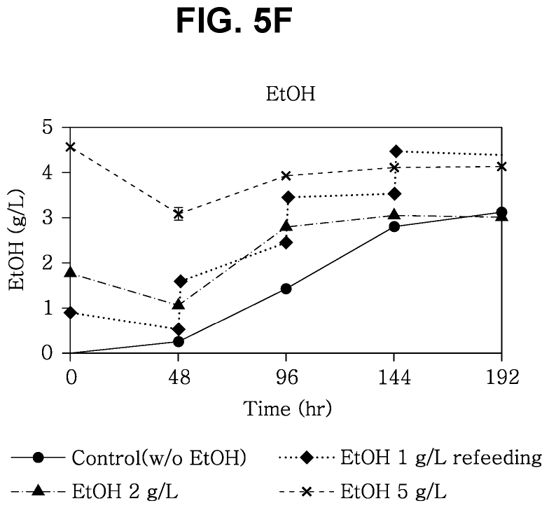

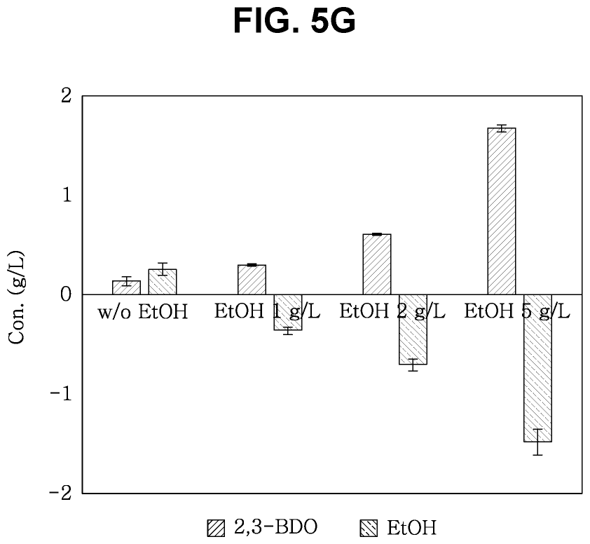

As a result, whereas 0.32 g/L of 2,3-butanediol was produced in 336 hours when the interval of the addition of synthesis gas was long (48 hours) (FIGS. 4A and 4D), 1.62 g/L of 2,3-butanediol was produced within only 192 hours when the interval of the addition of synthesis gas was short (24 hours) (FIGS. 5A and 5E). Therefore, it was confirmed that the production of 2,3-butanediol was increased as the addition amount of carbon monoxide was increased through continuous addition of synthesis gas.

Through this, it was confirmed that, even when the composition and addition amount of the synthesis gas are same, the production of 2,3-butanediol can be improved by maintaining the amount of carbon monoxide in the synthesis gas. Therefore, experiment was performed by reducing the interval of the addition of synthesis gas when investigating the production of 2,3-butanediol depending on the addition amount of ethanol.

### [Experimental Example 4-2] Change in Production of 2,3-Butanediol Depending on Addition Amount of Ethanol

First, it was investigated whether the productivity of 2,3-butanediol is improved if the addition amount of ethanol is increased in the early stage of fermentation. In addition, since the addition of excess ethanol in the early stage of fermentation may inhibit microbial growth, it was investigated whether the productivity of 2,3-butanediol can be improved with reduced inhibition by ethanol by adding low-concentration ethanol during the fermentation.

Fermentation was conducted using C. autoethanogenum DSMZ 10061 in the same manner as in Experimental Example 4-1, except that 1 g/L (hereinafter, Example 2-2) (EtOH 1 g/L refeeding in FIGS. 5B, 5E and 5F), 2 g/L (hereinafter, Example 2-3) (EtOH 2 g/L in FIGS. 5C, 5E and 5F) or 5 g/L (hereinafter, Example 2-4) (EtOH 5 g/L in FIGS. 5D-5F) of ethanol was added to the medium in the early stage of fermentation. For Example 2-2, a total of 4 g/L of ethanol was added to the medium, by 1 g/L each, while continuously supplying synthesis gas before carbon monoxide was consumed completely, specifically when the partial pressure was higher than 0 kPa, more specifically 5 kPa or higher. For Example 2-1, ethanol was not added (Control in FIG. 5A, Control (w/o EtOH) in FIGS. 5E and 5F). The result is shown in FIGS. 5A-5F.

As shown in FIGS. 5A-5D, carbon monoxide and hydrogen were consumed fast in the early stage of fermentation regardless of the addition amount of ethanol. The amount of carbon monoxide consumed during the fermentation was 153.11 kPa for Example 2-1, 145.44 kPa for Example 2-2 (1 g/L of ethanol was added repeatedly), 148.56 kPa for Example 2-3 (2 g/L of ethanol was added) and 128.81 kPa for Example 2-4 (5 g/L of ethanol was added). In particular, the consumption of carbon monoxide was decreased as the addition amount of ethanol was increased. The amount of carbon monoxide during the fermentation was decreased by 24.3 kPa for Example 2-4 (5 g/L of ethanol was added), which corresponds to 15.87% as compared to that of Example 2-1. Through this, it was confirmed that it is necessary to consider bacterial growth when adding 5 g/L or more of ethanol. In addition, the addition of acetate or ethanol did not decrease the consumption of carbon monoxide.

In addition, as shown in FIGS. 5E-5F, after 48 hours of fermentation, 0.13 g/L of 2,3-butanediol was produced for Example 2-1 (ethanol was not added) and 0.29 g/L of 2,3-butanediol was produced for Example 2-2 (1 g/L of ethanol was added). Also, after 48 hours of fermentation, 0.60 g/L of 2,3-butanediol was produced for Example 2-3 (2 g/L of ethanol was added) and 1.67 g/L of 2,3-butanediol was produced for Example 2-4 (5 g/L of ethanol was added). For Example 2-4 (5 g/L of ethanol was added), the production of 2,3-butanediol was improved 12.85 times as compared to Example 2-1 (ethanol was not added) (FIG. 5G).

Through this, it was confirmed that the production of 2,3-butanediol is increased as the addition amount of ethanol is increased.

Meanwhile, referring to FIGS. 5F and 5G, 3.14 g/L (ethanol production=ethanol concentration at 192 hours-ethanol concentration at 1 hour) of ethanol was produced at the end of the fermentation for Example 2-1 (ethanol was not added, Control (w/o EtOH)), whereas the amount of added ethanol was decreased by 32.31-40.66% after 48 hours of fermentation for Examples 2-2 to 2-4 wherein ethanol was added (FIG. 5G). After 48 hours, 1.40 g/L and 1.25 g/L of ethanol was produced for Example 2-2 (1 g/L of ethanol was added repeatedly) and Example 2-3 (2 g/L of ethanol was added), respectively. This suggests that the production efficiency of 2,3-butanediol is decreased because the reducing power produced during the metabolism of synthesis gas, which is a carbon source, is consumed for the production of both 2,3-butanediol and ethanol. In contrast, for Example 2-4 (5 g/L of ethanol was added), the ethanol concentration 4.58 g/L in the early stage of fermentation was decreased to 4.16 g/L at the end of fermentation. Through this, it was confirmed that, although the addition of high-concentration ethanol in the early stage of fermentation inhibits the consumption of carbon monoxide, the productivity of 2,3-butanediol is improved as ethanol is decreased in the early stage of fermentation and the production of 2,3-butanediol is improved.

In conclusion, it was confirmed that 2,3-butanediol can be produced without genetic engineering of microorganisms, use of catalysts, etc. by continuously adding synthesis gas to a medium to which a 2,3-butanediol-producing bacterium has been inoculated and, in particular, that the productivity of 2,3-butanediol can be improve by controlling the amount of carbon monoxide in the synthesis gas, the supply amount of the synthesis gas and the stirring speed of the medium. In addition, it was confirmed that 2,3-butanediol can be prepared by adding ethanol to the medium while continuously adding synthesis gas and that the productivity of 2,3-butanediol can be improved by adjusting the addition amount of ethanol.

1. A medium composition for preparing 2,3-butanediol, wherein
the composition comprises ethanol as an active ingredient,
the 2,3-butanediol is prepared from synthesis gas, and
the composition is for culturing a 2,3-butanediol-producing bacterium.

2. The medium composition for preparing 2,3-butanediol according to claim 1, wherein the 2,3-butanediol-producing bacterium is one or more selected from a group consisting of Clostridium hungdahlii, Clostridium autoethanogenum, Clostridium ragsdalei, Clostridium coskatii, Eubacterium limosum, Clostridium carboxidivorans P7, Peptostreptococcus productus and Butyribacterium methylotrophicum.

3. The medium composition for preparing 2,3-butanediol according to claim 1, wherein the content of the ethanol is 1-25 g/L based on the total volume of a medium comprising the medium composition.

4. The medium composition for preparing 2,3-butanediol according to claim 1, wherein the synthesis gas comprises carbon monoxide, carbon dioxide and hydrogen.

5. The medium composition for preparing 2,3-butanediol according to claim 1, wherein the synthesis gas is added continuously.

6. A method for preparing 2,3-butanediol, comprising:
a step of inoculating a 2,3-butanediol-producing bacterium to a medium comprising the composition according to claim 1; and
a step of adding synthesis gas to the medium.

7. The method for preparing 2,3-butanediol according to claim 6, wherein the addition amount of synthesis gas is 0.2-5 bar.

8. The method for preparing 2,3-butanediol according to claim 6, wherein the synthesis gas is added such that carbon monoxide exists at 1 kPa or higher.

9. The method for preparing 2,3-butanediol according to claim 6, wherein the method further comprises a step of stirring the medium to which the synthesis gas has been added, and the stirring is performed at a speed of 50-1000 rpm.

10. The method for preparing 2,3-butanediol according to claim 6, wherein the method further comprises a step of further adding 0.2-5 g/L of ethanol based on the total volume of the medium once or more times.
